(19) United States
(12) Patent Application Publication
(10) Pub. No.: US 2011/0092708 A1
(43) Pub. Date: Apr. 21, 2011

(54) PROCESS FOR THE PREPARATION OF MONTELUKAST AND ITS SALTS THEREOF

(76) Inventors: Seeta Ramanjaneyulu Gorantla, Hyderabad (IN); Mohan Bandari, Hyderabad (IN); Venkata Sunil Kumar Indukuri, Hyderabad (IN); Srinivas Simhadri, Hyderabad (IN); Narasimha Rao Ketavarapu, Hyderabad (IN); Venkata Krishna Kishore Jammulaveera, Hyderabad (IN); Yaseen Mohammed, Hyderabad (IN)

(21) Appl. No.: 12/376,354

(22) PCT Filed: Jul. 31, 2007
(86) PCT No.: PCT/IN2007/000314

§ 371 Date: Feb. 4, 2009

(30) Foreign Application Priority Data
Aug. 4, 2006 (IN) 1400CHE2006

(51) Int. Cl. C07D 215/18

(52) U.S. Cl. 546/174

(57) ABSTRACT

The present invention relates to the alkyl amine salts of Montelukast and methods for their preparation and conversion of Montelukast amine salts to Montelukast or Montelukast alkali/alkaline salt.

### FIELD OF THE INVENTION

The present invention relates to Montelukast, its alkaline salts using novel primary amine salts of 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid and the process for preparation thereof.

### BACKGROUND OF THE INVENTION

Montelukast sodium namely Sodium 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid has the formula

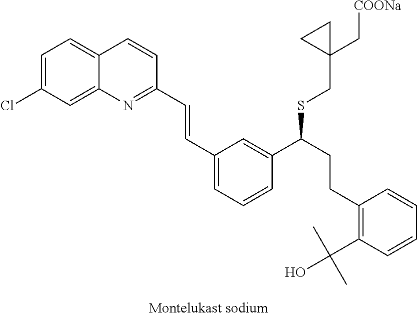

Montelukast sodium is a leukotriene antagonist and inhibits the synthesis of leukotriene biosynthesis. It is useful as anti-asthmatic, anti-allergic, anti-inflammatory, cytoprotective agent and hence useful in the treatment of angina, cerebral spasm, glomerular nephritis, hepatic, end toxemia, uveitis and allograft rejection.

EP 0480717 discloses Montelukast sodium along with other related compounds and the methods for their preparation. The reported method of synthesis proceeds through corresponding methyl ester namely, Methyl 2-[(3S)-[3-[(2E)-(7-chloroquinolin-2-yl)ethenyl]phenyl]-3-hydroxypropyl]benzoate and involves coupling methyl 1-(mercaptomethyl)cyclopropane acetate with a mesylate generated in-situ. The methyl ester is hydrolyzed to free acids and the latter converted directly to Montelukast sodium salt (Scheme-1). The process is not suitable for large-scale production because it requires tedious chromatographic purification of the methyl ester intermediate and/or the final product with low yield.

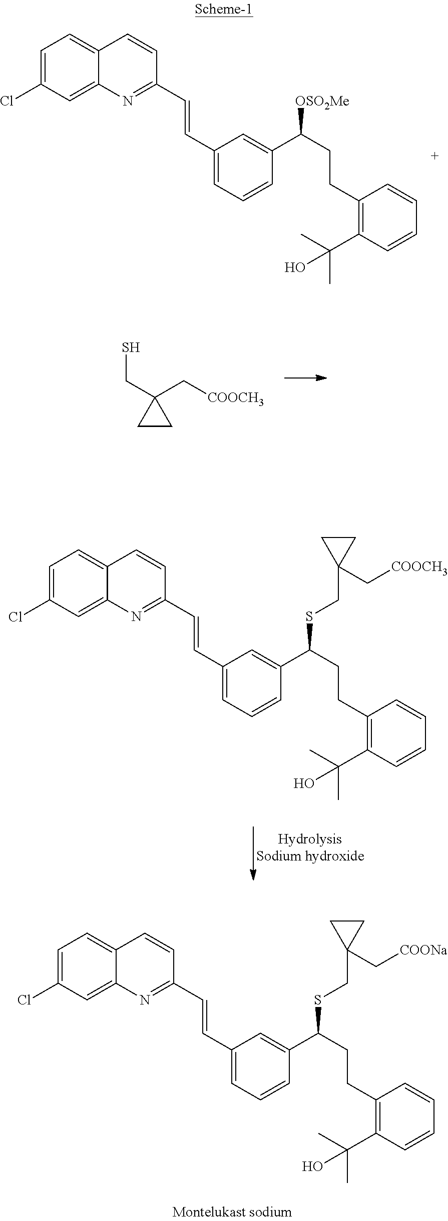

U.S. Pat. No. 5,614,632 discloses that the products obtained as per EP 0,480,717 are amorphous sodium salts, which are hydrated and often not ideal for pharmaceutical formulation and therefore provided an improved process for the preparation of crystalline Montelukast sodium, which involves isolation of Montelukast as its dicyclohexyl amine salt and converting it to Montelukast sodium.

WO 2006/08751 discloses a process for preparation of Montelukast sodium by neutralizing Montelukast organic amine salts such as α-Methyl benzyl amine salt, diisopropyl amine salt, dibenzyl amine salt followed by treatment with ethanolic sodium hydroxide.

US 2005/107612 discloses a process for preparation of Montelukast sodium by the neutralization of Montelukast amine salts such as tert butyl amine salt and phenyl ethyl amine salt followed by treatment with ethanolic sodium hydroxide.

Our co pending application 872/CHE/2005 discloses a process for preparation of Montelukast sodium by the neutralization of Montelukast amine salts such as (S) Cyclohexyl ethyl amine salt and Diisopropyl amine salt followed by treatment with ethanolic sodium hydroxide.

### SUMMARY OF THE INVENTION

The main object of the present invention is to provide novel primary amine salts of Montelukast.

Another object of the invention is to provide a process for the preparation of novel primary amine salts of Montelukast.

Another object of the present invention is to provide a process for the preparation of Montelukast sodium using primary amine salts of Montelukast.

Another object of the present invention is to provide a process for the preparation of Montelukast free acid using primary amine salts of Montelukast.

Accordingly, the present invention relates to the primary amine salts of Montelukast, method for the preparation and conversion to Montelukast/Montelukast sodium.

As illustrated in Scheme-2, condensation of Methyl 2-[(3S)-[3-[(2E)-(7-chloroquinolin-2-yl)ethenyl]phenyl]-3-halopropyl]benzoate with 1-(mercaptomethyl)cyclopropane acetic acid in presence of alkali hydride/alkali or alkaline carbonate/alkoxide affords 2-[1-[1(R)-[3-[2-(7-chloroquinolin-2-yl)ethenyl]phenyl]-3-[2-(methoxycarbonyl)phenyl]propyl sulfanylmethyl]cyclopropane acetic acid, isolated as amine salts which upon neutralization followed by reaction with Grignard reagent (methyl magnesium chloride or methyl magnesium bromide) in presence of cerium chloride affords the Montelukast, can be isolated as Montelukast free acid or Treating with primary amines results the Montelukast primary amine salts.

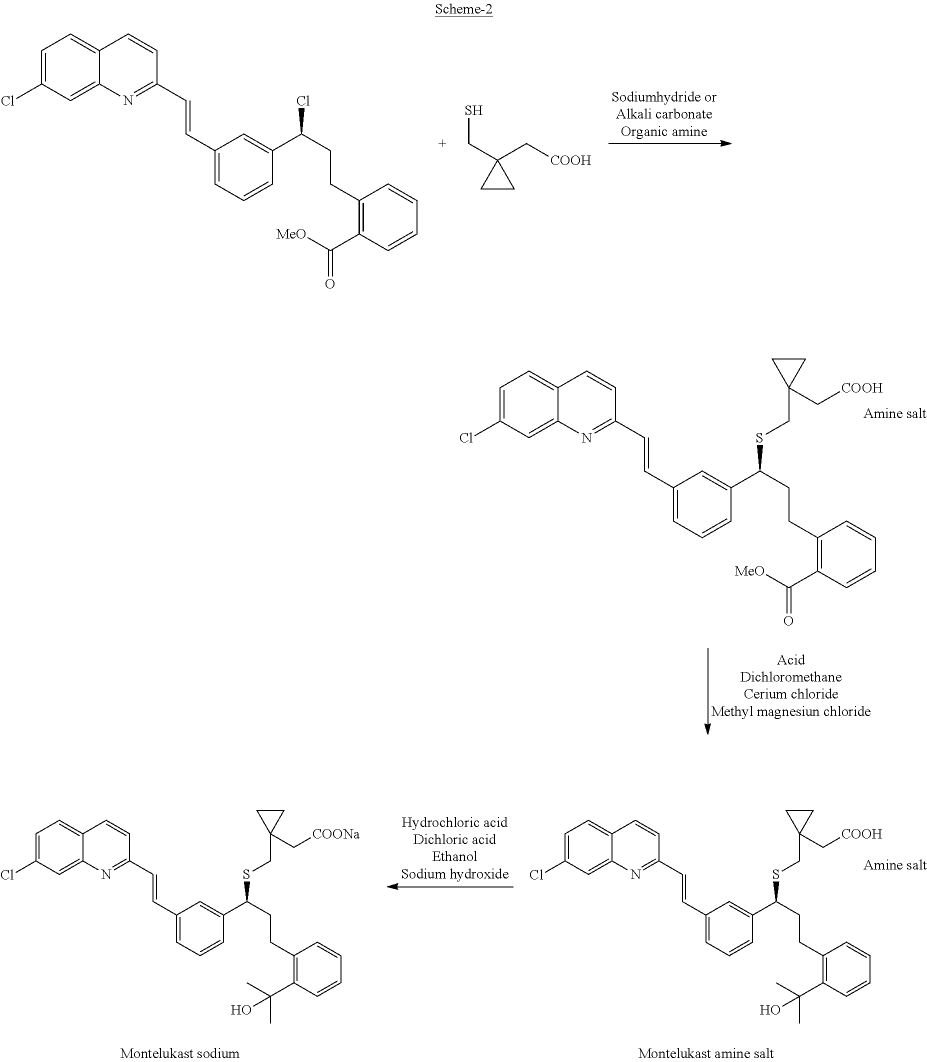

In another embodiment as illustrated in Scheme-3, reacting Methyl 2-[(3S)-[(3S)-[(2E)-(7-chloro quinolin-2-yl)ethenyl]phenyl]-3-halopropyl]benzoate with Grignard reagent (methyl magnesium chloride or methyl magnesium bromide) in presence of cerium chloride affords 2-[2-[3S-[3-[2-(7-chloroquinolin-2-yl)ethenyl]phenyl]-3-halopropyl]phenyl]-2-propanol which upon condensation with 1-(mercapto methyl)cyclopropane acetic acid in presence of alkali hydride or alkoxide or carbonates followed by saltification with primary amines results the Montelukast primary amine salts. The Montelukast amine salts can be converted to Montelukast sodium or optionally to Montelukast free acid.

Montelukast primary amine salts can also be prepared by treating the Montelukast free acid with primary amine in a solvent. The primary amine salts of Montelukast are also prepared by condensation of 2-[2-[3(s)-[3-(2-(7-chloro quinolin-2-yl)ethenyl]phenyl]-3-methane sulfonyloxy propyl]phenyl]-2-propanol with dilithium dianion of 1-(mercaptomethyl)cyclopropane acetic acid followed by treatment with primary amine.

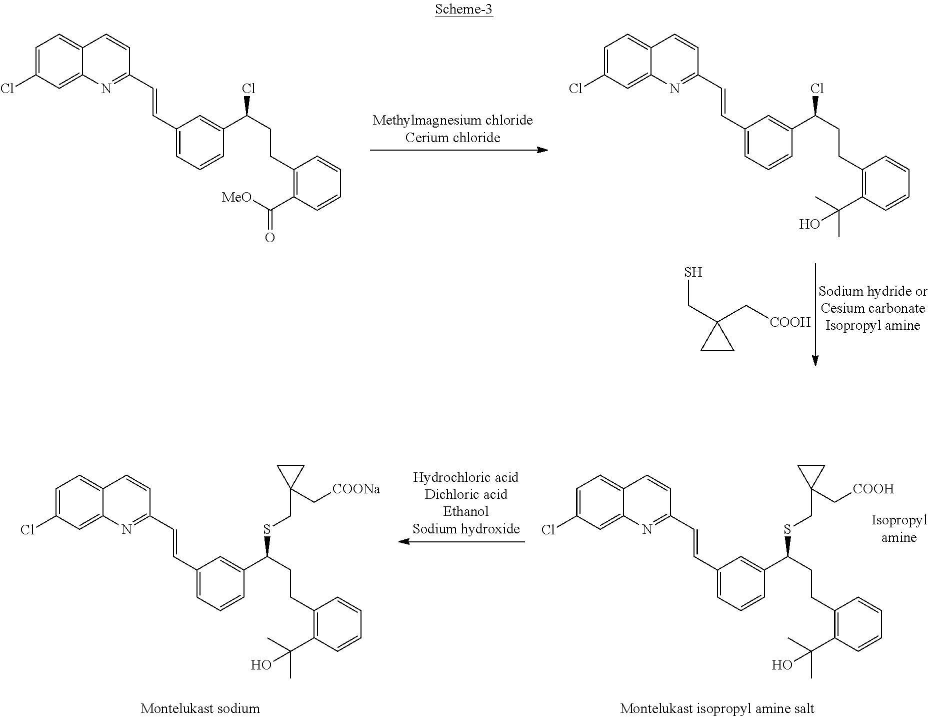

Montelukast amine salts can be purified and converted to Montelukast free acid or alkali/alkaline salts such as Montelukast sodium by following the procedure reported in the literature.

### BRIEF DESCRIPTION OF THE DRAWINGS

FIG. 1 X-Ray diffraction pattern of the Montelukast isopropyl amine salt

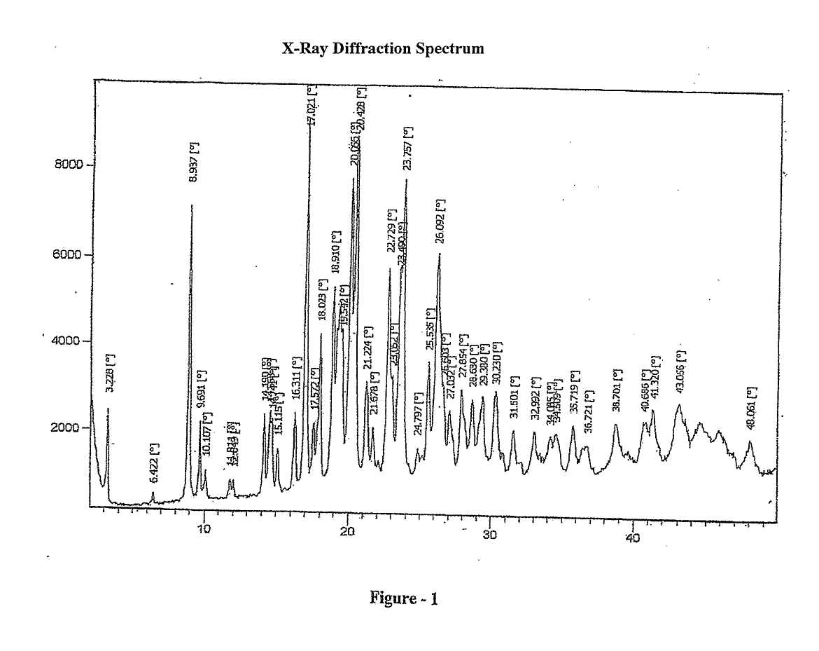

FIG. 2 FTIR spectrum of the Montelukast isopropyl amine salt

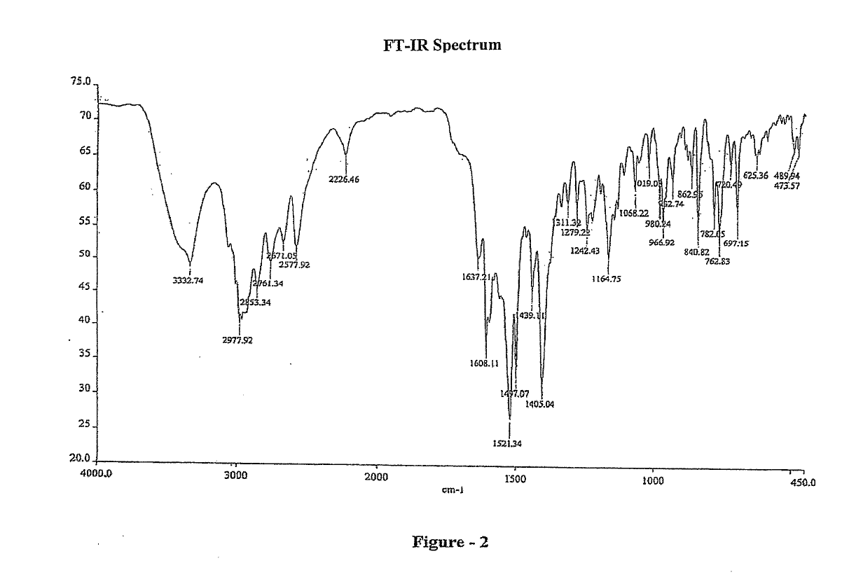

FIG. 3 X-ray diffraction pattern of the Montelukast Cyclohexyl amine salt

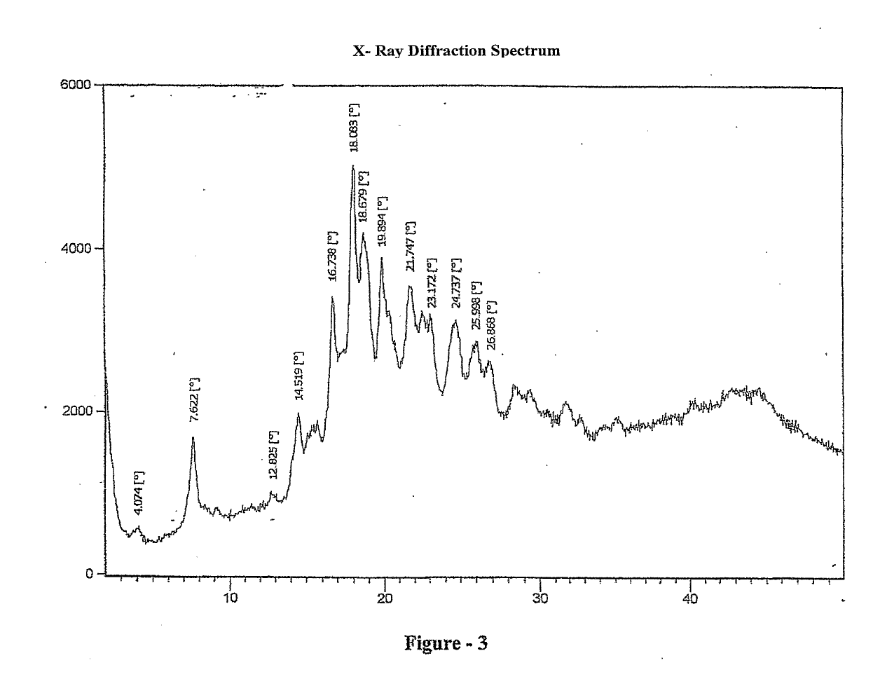

FIG. 4 FTIR spectrum of the Montelukast cyclohexyl amine salt

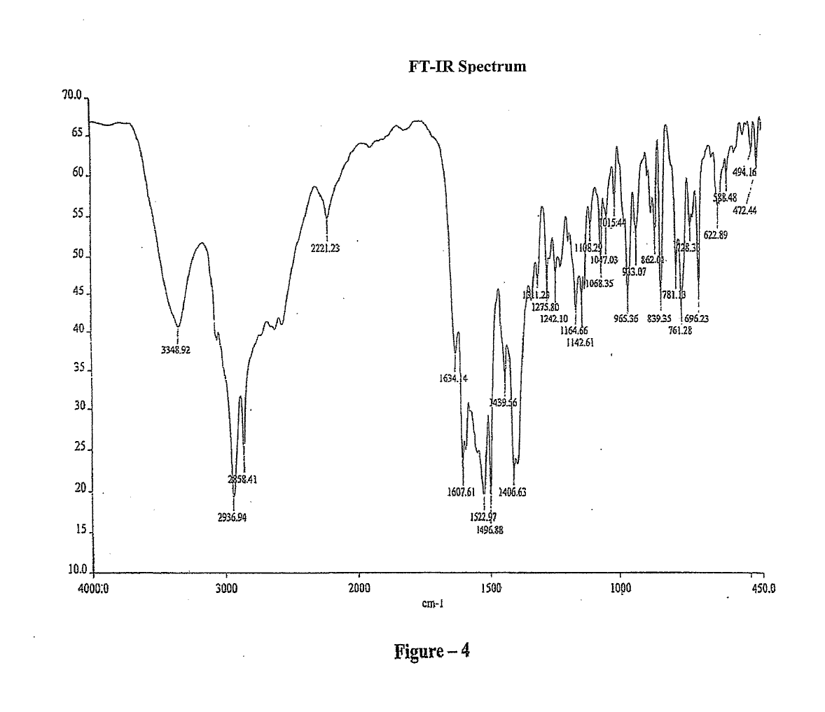

### DETAILED DESCRIPTION OF THE INVENTION

The process of the present invention comprising the steps of:

Condensation of Methyl 2-[(3S)-[3-[(2E)-(7-chloro quinolin-2-yl)ethenyl]phenyl]-3-halopropyl]benzoate with 1-(mercapto methyl)cyclopropane acetic acid in presence of alkali hydride, alkoxide or carbonate to give 2-[1-[1(R)-[3-[2-(7-chloroquinolin-2-yl)ethenyl]phenyl]-3-[2-(methoxy carbonyl)phenyl]propylsulfanylmethyl]cyclopropane acetic acid which can be isolated as primary amine salts.

Reacting 2-[1-[1(R)-[3-[2-(7-chloroquinolin-2-yl)ethenyl]phenyl]-3-[2-(methoxy carbonyl)phenyl]propylsulfanylmethyl]cyclopropane acetic acid with Grignard reagent to give Montelukast.

Isolating Montelukast as Montelukast primary amine salt

Converting Montelukast primary amine salt to Montelukast free acid and or it's required alkali/alkaline salt.

OR

Condensing 2-[2-[(3S)-[3-[(2E)-(7-chloroquinolin-2-yl)ethenyl]phenyl]-3-chloro propyl]phenyl-2-propanol with 1-(mercapto methyl)cyclopropane acetic acid in presence of alkali hydride or alkoxide or carbonate to give Montelukast.

Isolating Montelukast as Montelukast primary amine salt.

Converting Montelukast primary amine salt to Montelukast free acid and or its required alkali/alkaline salt

OR

Condensation of 2-[2-[3(s)-[3-(2-(7-chloro quinolin-2-yl)ethenyl]phenyl]-3-methane sulfonyloxy propyl]phenyl]-2-propanol with dilithium dianion of 1-(mercapto methyl)cyclopropane acetic acid to give Montelukast,

Isolating Montelukast as Montelukast primary amine salt.

Converting Montelukast primary amine salt to Montelukast free acid and or its required alkali/alkaline salt

In a specific embodiment, the present invention provides a process for the preparation of Montelukast primary amine salt, which involves;

Condensing 1-(mercaptomethyl)cyclopropane acetic acid with 2-[2-[(3S)-[3-(2E)-(7-chloroquinolin-2-yl)ethenyl]phenyl]-3-halopropyl]phenyl-2-propanol resulting 2-[1-[1(R)-[3-[2-(7-chloroquinolin-2-yl)ethenyl]phenyl]-3-[2-(methoxycarbonyl)phenyl]propylsulfanylmethyl]cyclopropane acetic acid and isolating as its amine salt preferably isopropyl amine salt or cyclohexylamine salt

Adding methyl magnesium chloride to a suspension of cerium chloride in tetrahydrofuran slowly at temperature of −5° C. to 5° C. and maintaining at −5° C. to 5° C. for about 2 hrs,

adding a solution of 2-[1-[1(R)[3-[2-(7-chloroquinolin-2-yl)ethenyl]phenyl]-3-[2-(methoxycarbonyl)phenyl]propylsulfanylmethyl]cyclopropane acetic acid in toluene at temperature of −5° C. to 5° C.,

maintaining for about 2 to 8 hrs and quenching the reaction mass into a mixture of dilute acetic acid and alkyl ester solvent such as methyl acetate, ethyl acetate, isopropyl acetate, butyl acetate at temperature below 20° C.,

separating the layers, washing the organic layer with sodium carbonate solution followed by drying over dehydrating agents, concentrating the organic layer and adding the above used alkyl ester solvent

treating with alkyl amines at temperature 10° C. to 30° C. followed by maintaining for about 12 hrs to 48 hrs,

Adding C₅ to C₇ hydrocarbon solvent such as hexane, heptane, cyclohexane, methyl cyclohexane, toluene and stirring for another 8 to 24 hrs yields the Montelukast alkyl amine salt.

In another preferred embodiment, the present invention provides a process for the preparation of Montelukast amine salt, which involves

Adding 1-(mercaptomethyl)cyclopropane acetic acid in dimethyl formamide to a suspension of alkali hydride or alkali alkoxide or alkali carbonate (the preferable alkali hydrides are sodium hydride, alkoxides are sodium methoxide, potassium tertiary butoxide, alkali carbonates are cesium carbonate) in dimethylformamide/tetrahydrofuran at temperature of −10° C. to 100° C., preferably about −5° C. to 70° C.,

stirring for about 30 min. to 3 hrs and slowly adding a solution of 2-[2-[(3S)-[3-[(2E)-(7-chloroquinolin-2-yl)ethenyl]phenyl]-3-halopropyl]phenyl-2-propanol in dimethylformamide over 30 min. to 4 hrs at temperature of −10° C. to 30° C. preferably about −5° C. to 10° C. and stirring for about 10 hrs to 24 hrs,

quenching the reaction mass to a mixture of water and alkyl ester solvent such as methyl acetate, ethyl acetate, isopropyl acetate.

separating the layers, extracting the aqueous layer with ethyl acetate and washing the combined organic layer successively with tartaric acid solution and 5% NaCl solution.

drying the organic layer over dehydrating agents and concentrating the solution, adding the above used alkyl ester solvent

treating with alkyl amines at temperature 10° C. to 30° C. and maintaining for about 12 to 48 hrs,

Adding the C₅ to C₇ hydrocarbon solvent such as hexane, heptane, cyclohexane, methyl cyclohexane, toluene and stirring for another 8 hrs to 24 hrs yields the Montelukast primary amine salt(s).

The prepared Montelukast primary amine salts are Montelukast primary amine salts preferably isopropyl amine salt and cyclohexylamine salt which are found to be novel and characterized by chemical analysis, NMR, Mass and IR spectral data.

Montelukast primary amine salt(s) can be purified (if required) and converted to required Montelukast alkali/alkaline salts preferably sodium salt as described below;

Suspending the Montelukast primary amine salt(s) in a mixture of water and water immiscible solvent such as toluene, methylene chloride,

acidifying with organic acid such as acetic acid, or inorganic acid such as hydrochloric acid, sulphuric acid.

separating the layers, washing the organic layer with water,

drying over dehydrating agents,

adding the ethanolic sodium hydroxide solution to the dried organic layer,

removing the solvents preferably under reduced pressure at temperature below 40° C. to gives the residue

Adding second solvent such as alkyl hydrocarbons like heptane or ether solvent such as diisopropyl ether, diethyl ether

Isolating and drying at 30°-100° C. affords Montelukast sodium preferably under vacuum.

Montelukast alkyl amine salt(s) can be purified (if required) and converted to Montelukast free acid as described below;

Suspending the Montelukast primary amine salt(s) in a mixture of water and methylene chloride,

adding dilute acetic acid or dil.HCl, separating the layers,

washing the organic layer with water, drying over dehydrating agents, removing the methylene chloride under reduced pressure at temperature below 40° C.,

dissolving the residue in ethyl acetate or Toluene or methanol or ethanol or acetone or dichloromethane at 50-60° C. followed by gradual cooling 20° C. to 25° C.,

Isolating the product and drying at 40° C. to 50° C. gives the Montelukast free acid.

Montelukast alkyl amine salt(s) can be prepared from Montelukast free acid by;

dissolving the Montelukast free acid in alkyl ester solvent such as methyl acetate, ethyl acetate, isopropyl acetate adding the alkyl amine at temperature of 20° C.-35° C.

Maintaining the mass for about 10 to 36 hrs, adding the second solvent selected from hydrocarbon of C-5 to C-7, acetonitrile, ethers of C-4 to C-8 and maintaining for about 2 to 18 hrs, The preferred hydrocarbon is n-Hexane, n-Heptane, toluene, cyclohexane, methyl cyclohexane and the preferred ethers is diethyl ether or diisopropyl ether.

Isolating the product and drying yields Montelukast primary amine salt

According to the present invention, Montelukast is isolated as primary amine salts such as cyclohehyl amine and isopropyl amine with improved yield and quality. Montelukast amine salts are converted to Montelukast free acid, Montelukast sodium salts with pharmaceutically acceptable grade with improved yield.

The invention is now illustrated with a few non-limiting examples.

### Example-I

### Preparation of 2-[1-[1-[1(R)-[3-[2-(7-Chloroquinolin-2-yl)ethenyl]Phenyl]-3-[2-(methoxycarbonyl)phenyl]propylsulfanylmethyl]cyclopropyl]acetic acid isopropyl amine salt

Suspend Sodium hydride (28 Gm, 0.70 moles) in DMF (400 ml), cool to −5° C. under nitrogen, slowly add the solution of 1-(mercaptomethyl)cyclopropane acetic acid (46 Gms, 0.315 mole) in DMF (100 ml) at −5° C. to 0° C. over 1 hr and maintain at −5° C. to 0° C. for 1 hr. Then slowly add Methyl 2-[(3S)-[3-[(2E)-(7-chloro quinolin-2-yl)ethenyl]phenyl]-3-chloropropyl]benzoate (100 Gms, 0.21 mole) in 4 equal lots at −5° C. to 0° C. over 1 hr and maintain the reaction mass at −5° C. to 0° C. for 24 hrs. Transfer the reaction mass into a mixture of 5% NaCl solution (1000 ml): ethyl acetate (1000 ml) and mix for 30 min. at temperature below 20° C. pH of the reaction mass is adjust to 7.0 by addition of 20% aqueous solution of Tartaric acid (100 ml) at 10° C.-25° C. and mix for 30 min. Allow to settle the layers, separate the organic layer and extract the aqueous layer with ethyl acetate (1000 ml). Combine the organic layer and ethyl acetate extraction, wash with 5% aqueous tartaric acid solution (400 ml), 5% NaCl solution (2×1000 ml) twice, dry over sodium sulphate, treat with carbon for 30 min at 25° C.-35° C. and distill off ethyl acetate under reduced pressure at temperature below 40° C. to get the residue. Dissolve the residue in ethyl acetate (600 ml) by heating to 45° C., cool to 20° C. under N₂ atmosphere and slowly add the isopropyl amine (15 Gms) over 30 min. at 20° C. Maintain at 20° to 22° C. for 1 hr, seed with 2-[1-[1(R)-[3-[2-(7-Chloroquinolin-2-yl)ethenyl]Phenyl]-3-[2-(methoxycarbonyl)phenyl]propylsulfanylmethyl]cyclopropyl]acetic acid isopropyl amine salt (200 mg) and maintain at 20° C.-25° C. for 36 hrs under N₂ atmosphere. Slowly add n-Hexane (1200 ml) over 40 min, mix the reaction mass for 24 hrs at 20° C.-25° C. Filter the solid, wash with n-Hexane (500 ml) and dry at 40° C.-45° C. till constant weight.

Yield: 100 gms

### Example-II

### Preparation of 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid cyclohexyl amine salt (Montelukast CHA salt)

Step-1: Suspend 2-[1-[1(R)-[3-[2-(7-Chloroquinolin-2-yl)ethenyl]phenyl]-3-[2-(methoxy carbonyl)phenyl]propylsulfanylmethyl]cyclopropyl]acetic acid isopropyl amine salt (140 Gms, 0.21 mole) in a mixture of methylene chloride (1680 ml), water (980 ml) and mix for 15 min. Adjust the pH of the reaction mass to 4.5 with of 6% acetic acid (240 ml) at 25° C.-35° C., mix for 30 min, allow settling the layers, separating the organic layer and extracting the aqueous layer with methylene chloride (1000 ml). Combine the organic layers, wash with water (980 ml), dry over sodium sulphate and distill off methylene chloride initially atmospherically, finally under reduced pressure to get the residue. Dissolve the residue in toluene (1000 ml) and use the solution in next step.

Step-2: Raise the temperature of the suspension of cerium chloride (50 Gms) in THF (1050 ml), distill off initially 50 ml of THF and maintain the mass at reflux temperature (65° C.) for 3 hrs under nitrogen atmosphere. Cool the reaction mass to −5° C., add 3.0 Molar methyl magnesium chloride solution in THF (500 ml) at temperature −5° C.-0° C. over 40 min and maintain for 2 hrs at that temperature. Slowly add the step-1 solution over 60 min and maintain at 0° C.-5° C. for 30 min to 6 hrs. Transfer the reaction mass into a pre cooled mixture of 12% acetic acid (1400 ml): ethyl acetate (800 ml) at temperature below 20° C. and mix for 30 min at 18° C.-20° C. Allow to settle, separate the organic layer, extract the aqueous layer with ethyl acetate (800 ml), combine the organic layers, wash successively with 10% sodium carbonate solution (1600 ml), 5% sodium chloride solution (2×1000 ml) and dry the organic layer over anhydrous sodium sulphate (15 Gms). Treat the dried organic layer with carbon; distill off ethyl acetate from the clear solution at temperature below 45° C. under reduced pressure to get the residue. Add Ethyl acetate (500 ml) to the residue; raise the temperature to 45° C. and stir for 10 min. to get a clear solution. Gradually cool the reaction mass to 28° C.-32° C., Add Cyclohexylamine (Qty 25 gms) over 30 min, stir for 1 hr and seed with Montelukast Cyclohexylamine pure salt (500 mg) and maintain at 28° C.-32° C. for 12 hrs. Add n-heptane (1000 ml) over 1 hr. Stir the mass for 15 hrs. Filter the product, wash with n-heptane (50 ml) and dry at 45° C.-50° C. till constant weight.

The dry weight of the Montelukast CHA salt is 100 Gms

### Example-III

### Preparation of 2-[1-[1(R)-[3-[2-(7-Chloroquinolin-2-yl)ethenyl]phenyl]-3-[2-(1-hydroxy-1-methylethyl)phenyl]propyl sulfanyl methyl]cyclopropyl]acetic acid Isopropyl amine salt (Montelukast IPA salt)

Charged DMF (500 ml) and 2-[2-[3S[3-[2-(7-Chloroquinolin-2-yl)ethenyl]phenyl]-3-chloropropyl]phenyl]-2-propanol (100 gms) at 25-35° C. under nitrogen atmosphere. Reaction mass is maintained at room temperature for 10-20 minutes to get a clear solution. Reaction mass temp is raised to 35° C. and charged Cesium Carbonate (205.3 gms) at 33-35° C. Reaction mass is maintained at 33-35° C. for 5-15 min. 1-(Mercaptomethyl)cyclopropane acetic acid (33.7 gms in 200 ml of DMF) is added to the above solution at 33-37° C. over 4-5 hrs. Maintained the reaction mass at 33-37° C. for 30 minutes and checked for the reaction completion. Cooled the reaction mass to 25-30° C. and quenched into a mixture of Ethyl acetate and 5% Sodium chloride (1000 ml+1000 ml) below 30° C. over 30 minutes. Separated the organic layer and the aqueous layer is extracted with 1000 ml of Ethyl acetate. Combined the organic layers and washed with 400 ml of 5% Tartaric acid followed by 2×1000 ml of 5% Sodium chloride solution. Dried the organic layer over sodium sulphate and treated with activated carbon. Ethyl acetate is removed completely under vacuum below 45° C. to get residue.

The obtained residue is dissolved in Ethyl acetate (600 ml) at about 45° C. and cooled the mass to 20-25° C. Isopropyl amine (17.9 ml) is added to the mass at 20-25° C. over 30 minutes. Maintained for 1 hr at 20-25° C. and then seeded with pure Isopropyl amine salt. Maintained the mass at 20-25° C. for 3-12 hrs n-Heptane (1200 ml) is added slowly to the mass at 20-25° C. over 30 minutes. Maintained the mass at 20-25° C. for 3-4 hrs, filtered the product and washed the wet cake with 100 ml of n-Heptane. Dried the material at 45-50° C.

Yield: 96.5 Gms.

### Example-IV

### Preparation of Montelukast Free Acid from Montelukast IPA Salt

Montelukast IPA salt (100 Gms, 0.146 moles) is suspended in a mixture of methylene chloride (1200 ml), water (700 ml). 6% acetic acid (193 ml) is added at temperature of 25° C.-35° C. Reaction mass is stirred for 30 min and allowed to settle. Methylene chloride layer is separated and the aqueous layer is extracted with methylene chloride (700 ml). The combined organic layer is washed with water (700 ml) and dried over sodium sulphate. Methylene chloride is distilled off under reduced pressure to get residue and the residue is dissolved in Toluene (160 ml). Toluene layer is gradually cooled to 20° C.-25° C. Maintained at 20° C.-25° C. for 4 hrs and the product is filtered. The wet cake is washed with chilled Toluene (50 ml) and dried at 45° C.-50° C. till constant weight.

Dry wt of Montelukast free acid is 60 Gms (70.3%)

### Example-V

### Preparation of Montelukast Sodium from Montelukast Isopropyl Amine Salt

2-[1-[1(R)-[3-[2-(7-Chloroquinolin-2-yl)ethenyl]phenyl]-3-[2-(1-hydroxy-1-methylethyl)phenyl]propyl sulfanyl methyl]cyclopropyl]acetic acid Isopropyl amine salt (100 gms) is suspended in MDC (2.0 Lts) and DM water (1.0 Lt) at 25-30° C. under Nitrogen. Maintained at 25-30° C. for 15-30 min and cooled the mass to 10° C. Adjusted the pH of the mass to 4.0-4.5 by addition of 1N HCl at 10-15° C. Raised the mass temperature to 20° C. and maintained at 20-25° C. for 30 minutes under nitrogen. Organic layer is separated and the aqueous layer is extracted with MDC (1000 ml). Combined the organic layers and washed with DM water (1000 ml). Dried the organic layer over sodium sulphate and cooled to 10° C. under Nitrogen. Ethanolic NaOH (329 ml of 0.486 M) is added to the MDC layer at 8-12° C. under Nitrogen over 90 minutes. Maintained the reaction mass at 8-12° C. for 30 minutes. Treated the reaction mass with activated carbon. Filtered the carbon over hyflow bed and washed the bed with 200 ml of MDC at 10-15° C.

Distilled off the solvent completely under vacuum below 40° C. to get white sticky mass. Charged 1000 ml of methanol to above sticky mass and heat to 40° C. to make clear solution. Treated the reaction mass with activated carbon at 35-40° C. Filtered the carbon over hyflow bed and washed the bed with 200 ml of methanol at 35-40° C. Distilled off methanol completely under reduced pressure at temperature below 40° C. to get white foam mass.

Charged n-Heptane (100 ml) and distilled off below 40° C. to get solid. Charged n-Heptane (2.0 Lts) and maintained at 25-35° C. under Nitrogen for 2-4 hours till material became free. Filtered the product and washed the wet cake with n-Heptane (200 ml). Dried the wet cake at temperature of 50-55° C. under high vacuum for 4 hours and 20 hours at 90-95° C. for till moisture content comes to below 1.0%.

Yield: 85 Gms.

While the present invention has been described in terms of its specific embodiments, certain modifications and equivalents will be apparent to those skilled in the art and are intended to be included within the scope of the present invention.

We claim:

1. A process for the preparation of Montelukast primary amine salt which comprising the steps of:
a) reacting methyl 2-[(3S)-[3-[(2E)-(7-chloro quinolin-2-yl)ethenyl]phenyl]-3-halopropyl]benzoate with 1-(mercapto methyl)cyclopropane acetic acid in presence of base to give 2-[1-[1(R)-[3-[2-(7-chloroquinolin-2-yl)ethenyl]phenyl]-3-[2-(methoxy carbonyl)phenyl]propylsulfanylmethyl]cyclopropane acetic acid,
b) reacting 2-[1-[1(R)-[3-[2-(7-chloroquinolin-2-yl)ethenyl]phenyl]-3-[2-(methoxy carbonyl)phenyl]propylsulfanylmethyl]cyclopropane acetic acid with primary amine to give primary amine salt of 2-[1-[1(R)-[3-[2-(7-chloroquinolin-2-yl)ethenyl]phenyl]-3-[2-(methoxy carbonyl)phenyl]propylsulfanylmethyl]cyclopropane acetic acid,
c) subjecting Grignard reaction of 2-[1-[1(R)-[3-[2-(7-chloroquinolin-2-yl)ethenyl]phenyl]-3-[2-(methoxy carbonyl)phenyl]propylsulfanylmethyl]cyclopropane acetic acid with methyl magnesium halide to give Montelukast,
d) treating Montelukast with primary amine and
e) isolating Montelukast primary amine salt.

2. A process according to claim 1, wherein the primary amine is selected from isopropyl amine and cyclohexyl amine

3. A process according to claim 1, wherein the base is selected from sodium hydride, sodium methoxide, potassium methoxide, potassium tertiary butoxide, sodium carbonate, potassium carbonate, cesium carbonate.

4. A process according to claim 1, wherein methyl magnesium halide is selected from methyl magnesium chloride, methyl magnesium bromide, methyl magnesium iodide.

5. A process for the preparation of Montelukast primary amine salt which comprising the steps of:
a). subjecting Grignard reaction of methyl 2-[(3S)-[3-[(2E)-(7-chloroquinolin-2-yl)ethenyl]phenyl]-3-halopropyl]benzoate with methyl magnesium halide to give 2-[2-[3S-[3-[2-(7-chloroquinoline-2-yl)ethyl]phenyl]-3-halopropyl]phenyl]-2-propanol,
b). condensing 2-[2-[3 S-[3-[2-(7-chloroquinoline-2-yl)ethyl]phenyl]-3-halopropyl]phenyl]-2-propanol with 1-(mercapto methyl)cyclopropane acetic acid in the presence of base to give Montelukast,
c) treating Montelukast with primary amine and
d) isolating Montelukast primary amine salt.

6. A process according to claim 5, wherein the primary amine is selected from isopropyl amine and cyclohexyl amine.

7. A process according to claim 5, wherein the base is selected from sodium hydride, sodium methoxide, potassium methoxide, potassium tertiary butoxide, sodium carbonate, potassium carbonate, cesium carbonate.

8. A process according to claim 4, wherein methyl magnesium halide is selected from methyl magnesium chloride, methyl magnesium bromide, methyl magnesium iodide.

9. A process for the preparation of Montelukast primary amine salt which comprising the steps of:
a). reacting 2-[2-[3S-[3-[2-(7-chloroquinoline-2-yl)ethyl]phenyl]-3-halopropyl]phenyl]-2-propanol with dilithium dianion of 1-(mercapto methyl)cyclopropane acetic acid to give Montelukast,
b). treating Montelukast with primary amine and
c). isolating Montelukast primary amine salt.

10. A process according to claim 9, wherein the primary amine is selected from isopropyl amine and cyclohexyl amine

11. A process for the preparation of Montelukast free acid which comprising the steps of:
a). suspending Montelukast primary amine salt in a mixture of water and water immiscible organic solvent,
b). adjusting resulting solution of step a, pH to acidic with acid,
c). separating the water immiscible organic solvent,
d) concentrating the organic solvent to give residue,
e) treating residue with organic solvent and
f) isolating Montelukast, free acid.

12. A process according to claim 11, wherein the primary amine is selected from isopropyl amine and cyclohexyl amine

13. A process according to claim 11, wherein the water immiscible organic solvent is selected from dichloromethane, dichloroethane, ethyl acetate, toluene.

14. A process according to claim 11, wherein the acid is selected from acetic acid, hydrochloric acid, hydrobromic acid, sulphuric acid, phosphoric acid.

15. A process according to claim 11, wherein the organic solvent is selected from heptane, hexane, cyclohexane, toluene, methanol, ethanol, acetone, dichloromethane, ethyl acetate.

16. A process for the preparation of Montelukast sodium salt which comprising the steps of:
a). suspending Montelukast primary amine salt in a mixture of water and water immiscible organic solvent,
b). adjusting resulting solution of step a, pH to acidic with acid,
c). separating the water immiscible organic solvent,
d) treating the organic, solvent step c, with sodium base
d) concentrating the organic solvent from step d, to give residue,
e) treating residue with organic solvent and
f) isolating Montelukast sodium salt.

17. A process according to claim 16, wherein the primary amine is selected from isopropyl amine and cyclohexyl amine

18. A process according to claim 16, wherein the water immiscible organic solvent is selected from dichloromethane, dichloroethane, ethyl acetate, toluene.

19. A process according to claim 16, wherein the acid is selected from acetic acid, hydrochloric acid, hydrobromic acid, sulphuric acid, phosphoric acid.

20. A process according to claim 16, wherein the sodium base is selected from sodium hydroxide, sodium methoxide in alcohol.

21. A process according to claim 16, wherein the organic solvent is selected from heptane hexane, cyclohexane.
